(19) United States
(12) Patent Application Publication
(10) Pub. No.: US 2009/0169659 A1
(43) Pub. Date: Jul. 2, 2009

(54) PURIFICATION AND ENDOTOXIN-REMOVAL PROCESS

(75) Inventor: Mathias-Heinrich Kreuter, Witterswil (CH)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: VERITRON LIMITED, Blackfriars, London (GB)

(21) Appl. No.: 12/093,644

(22) PCT Filed: Nov. 14, 2006
(86) PCT No.: PCT/GB2006/004240

§ 371 Date: Oct. 3, 2008

(30) Foreign Application Priority Data
Nov. 15, 2005 (GB) 0523257.4

(51) Int. Cl. A61K 36/28

(52) U.S. Cl. 424/764

(57) ABSTRACT

A process for purifying an aqueous composition including a water-soluble contaminant having lipid groups, e.g. an endotoxin, comprises contacting the composition with a lipophilic component that forms a complex with the contaminant; a first removal step, of material having a size larger than the complex; and a second removal step, of the complex.

### FIELD OF THE INVENTION

This invention relates to a purification process, and in particular to a process for the removal of endotoxins from plant extracts.

### BACKGROUND OF THE INVENTION

The therapeutic and other uses of plant extracts have been understood for millennia. Increasingly sophisticated techniques have been used to extract valuable materials, especially oils, from herbs and other plants. The products are generally intended to be taken by mouth.

It is of course desirable to remove contaminants from plant extracts. U.S. Pat. No. 6,024,998 describes a process for the removal of undesirable lipophilic contaminants found in beverages and vegetable preparations. Such contaminants include pesticides and other toxic materials that are typically applied during plant growth and which can accumulate in the soil and which can be retained on the plant parts.

The process described in U.S. Pat. No. 6,024,998 comprises mixing the vegetable preparation with a lipophilic phase in which the contaminants are dissolved and thereby concentrated, followed by removal of this lipophilic phase, e.g. by filtration. In this way, the whole of the plant extract can be retained and the foreign materials removed.

WO03/101479 describes a therapeutic product which may contain a camomile extract. It is suggested that this extract may have anti-inflammatory properties that are useful in reducing inflammation when, as is preferred, the product is to be given by injection.

Endotoxins of the type found in cell walls are pyrogens that are undesirable components of an injectable formulation. A typical maximum regulatory limit is 75 Eunits/ml; an initial target of <100 Eunits/ml is desirable.

### SUMMARY OF THE INVENTION

It has now been appreciated that endotoxins are generally water-soluble materials that will not be removed selectively, if at all, by the procedure described in U.S. Pat. No. 6,024,998. It has however been appreciated that endotoxins have lipid groups that form complexes with lipophilic materials, and can be removed by an analogous procedure.

According to the present invention, in a process for purifying an aqueous composition comprising a water-soluble contaminant having lipid groups, the composition is contacted with a lipophilic component that forms a complex with the contaminant; there then follow a first removal step, of material having a size larger than the complex, and a second removal step, of the complex.

In the novel process, the second removal step is typically ultrafiltration, and removes the endotoxins that complex with the lipophilic component. The first filtration or other removal step is necessary, to remove larger components that will block the ultrafiltration process.

### BRIEF DESCRIPTION OF THE DRAWINGS

FIGS. 1A and 1B are each flow diagrams representing the steps involved in an embodiment of the invention.

### DESCRIPTION OF PREFERRED EMBODIMENTS

The lipophilic component used in the present invention can be the same as that described in U.S. Pat. No. 6,024,998. Whereas such a component can form relatively large drops of a lipophilic phase in which lipophilic contaminants are dissolved, a characteristic of the present invention is that such a material can also complex with lipid groups in a generally water-soluble molecule such as an endotoxin; the complex is of a size that can be removed by ultrafiltration but not by microfiltration that is sufficient to remove the drops. Therefore, while the materials used in this invention may be the same as those in the prior art, the procedure is necessarily different.

Endotoxins and also antigens are primarily carbohydrates having pendant protein and lipid groups; the presence of the lipid groups is sufficient to form a complex with a suitable lipophilic material, but does not compromise the generally water-soluble nature of the carbohydrate molecule. Such pyrogenic molecules may have an inflammatory effect, on injection, and they should therefore be removed as far as possible from an injectable medicament.

The present invention is particularly suited to the removal of undesirable components from camomile, for the preparation of a medicament as described in WO03/101479. The flower head (capitulum) of the camomile plant (Matricaria recutita) is composed of two parts, i.e. the yellow disc-shaped or tubular flowers or florets (flores tubiformis or tubiflorum) and the white radiating flowers or florets (flores ligutatea). The former is of particular interest. By means of the invention, a useful product can be obtained by separating the tubular flowers from other parts of the camomile head/plant, extraction of the separated yellow part in water, and isolation of the extract/removal of endotoxins. The invention is nevertheless applicable to any herb or other plant preparation; examples of such plants are given in U.S. Pat. No. 6,024,998, the content of which is herein incorporated by reference.

Lipophilic components suitable for use in the invention are also described in U.S. Pat. No. 6,024,998. This component may be of animal, vegetable, mineral or synthetic origin. It is preferably non-toxic. Examples of suitable materials include fats such as cocoa butter and coconut fat; oils such as neutral oils, sunflower oils, and fractionated coconut oil; waxes such as stearins, jojoba oil, beeswax, spermaceti and carnauba wax; paraffins, including vaseline; lipids; and sterols. All such compounds, whether pure or used as mixtures, preferably meet the requirements of the Deutsches Arzneibuch, the British Pharmacopoeia, the European Pharmacopeia or the US Food Chemical Codex. Particularly preferred materials are miglyol, diglycerides, triglycerides and ricinus oil. This last material includes ricinoleic acid, an example of a long-chain fatty acid containing a polar group.

The aqueous extract that may be subjected to a purification process according to the present invention typically comprises a multi-component mixture of water-soluble components. It may be obtained by adding water to the appropriate plant part, to obtain a suspension that is then usually heated to a temperature below the boiling point of water, e.g. 90-94° C., and then cooled to room temperature.

The aqueous extract is then subjected to the two filtration steps. For the purposes of illustration only, these will be described below as microfiltration and ultrafiltration, respectively. Other techniques, such as use of a lipophilic barrier, may be suitable. Each filtration step may be conducted in one, two or more than two stages, if desired.

As indicated above, microfiltration is applied in order to remove material that would otherwise compromise the effectiveness of the ultrafiltration step. Microfiltration may indeed remove contaminants, as described in U.S. Pat. No. 6,024,998. This typically involves using a filter having a pore size of at least 0.1 μm. The pore size used in the subsequent, ultrafiltration step is typically 0.001 to 0.01, e.g. up to 0.1, μm.

Each filtration step is preferably conducted by membrane separation, using synthetic membranes of materials such as glass, metal, ceramic or synthetic plastics. Materials suitable for microfiltration include polypropylene and polytetrafluorethyene. Materials suitable for ultrafiltration Include polyether sulfones and regenerated cellulose.

When two liquid phases are separated, this is preferably conducted by means of membrane technology. For this purpose, tubular or so-called “cross-flow” membranes are preferred.

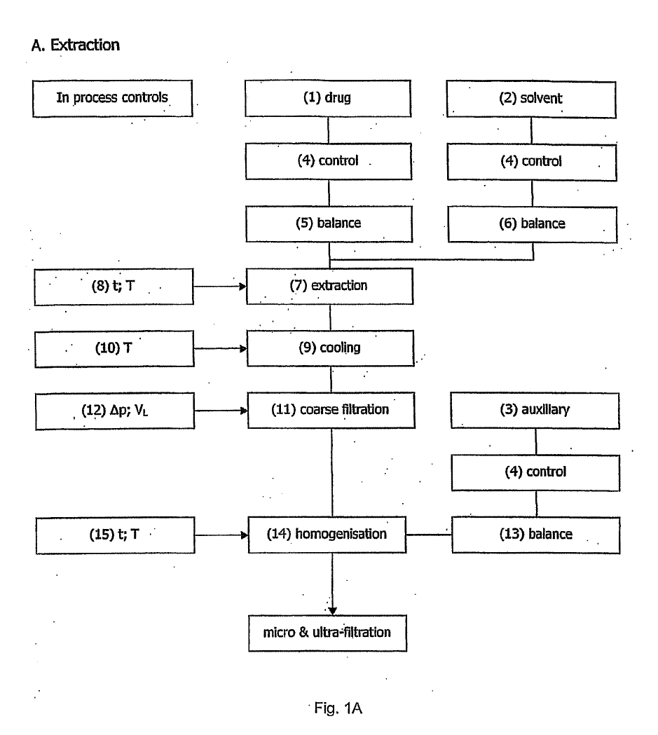

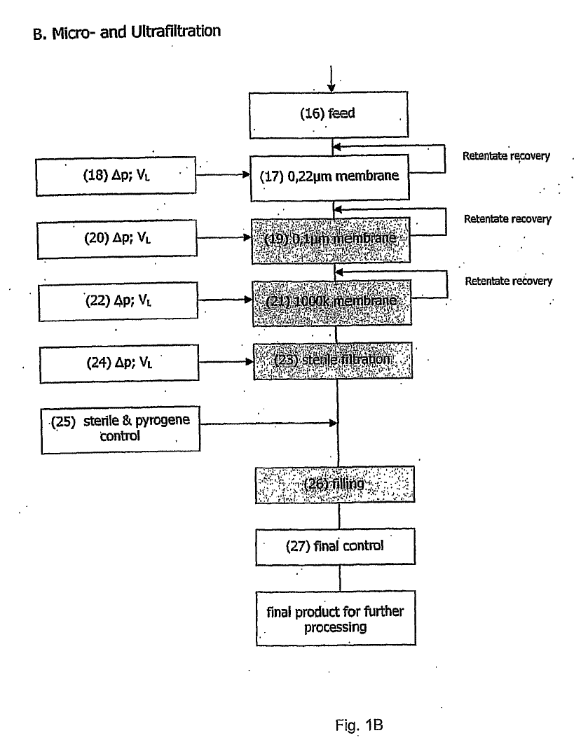

The product may be intended for use in therapy. It should then be sterile, and it is desirable that appropriate steps of its production should be conducted under sterile conditions. Such steps are these shown as 19, 21, 23 and 26, in FIG. 1B of the accompanying drawings. Such a procedure is illustrated in the following Examples 1 to 5. Example 6 also illustrates the invention, using a revised protocol. Examples 7 to 11 are comparative.

The experimental work reported below shows that the combination of a filtration cascade and the addition of a plant oil leads to a complete or nearly complete elimination of bacterial cell wall debris, known to a person skilled in the art as bacterial endotoxins or pyrogenes. These lipopolysaccharides or macromolecules are composed of a Lipid A moiety attached to a polysaccharide chain and are a major constituent of the cell wall of gram-negative bacteria. These complex macromolecules are water-soluble but surprisingly form high molecular complexes with plant oils resulting in a suspension and can be retained by molecular weight exclusion techniques, preferably by using ultrafiltration equipment. Molecular weight filtration microfiltration is of advantage to get rid of large piece of cell wall debris, mucilaginous cell wall fragments of the plant materials which would otherwise block the pores of ultrafiltration equipment.

The analysis of bacterial endotoxins of the samples obtained in the Examples was performed with the Cambrex PyroGene assay using a dilution factor of 1:10.000.

### Examples 1 and 2

45 g of yellow tubular camomile flowers (Chamomilla recutita) were mixed with 900 g of water (Aqua purificata, Ph. Helv.) This mixture was heated to a temperature between 90° C. and 94° C. within 20 to 30 minutes. Thereafter the mixture was stored at room temperature (15° C. to 25° C.) until a temperature between 30° C. and 35° C. was reached.

The drug residue was removed by deep layer filtration. The obtained crude filtrate was clarified by filtration through a 0.22 μm membrane.

To the clarified filtrate, 0.3% (Example 1) or 0.1% (Example 2), with respect to the extract mass, of ricinus oil (Ph. Eur. Grade) was added. The whole mixture was homogenised for 5 minutes. This prepared extract was filtered (in tangential flow mode) with retentate recovery via a 0.22 μm membrane.

The obtained permeate was filtered (in tangential flow mode) with retentate recovery via a 0.1 μm membrane, Finally, the obtained permeate was filtered (in tangential flow mode) with retentate recovery via a 1000 kDa membrane.

Residue of bacterial endotoxins in each final filtrate: <100 EU/ml

### Examples 3 to 5

Example 1 was repeated, except that, instead of ricinus oil, 0.3% (Example 3), 1.0% (Example 4) and 3.0% (Example 5), with respect to the extract mass, of mygliol (Ph. Eur.) was added to the clarified filtrate.

Residue of bacterial endotoxins in each final filtrate: <100 EU/ml.

### Example 6

This Example uses a revised protocol, in which heating and cooling were performed, not in an autoclave but in a 10 L double layer vessel under stirring (max. temperature of heating device 140° C.).

Miglyol was added instead of ricinus oil. The miglyol was “Miglyol 812 for parenteral use” from Hänseler. The mixture was stirred at room temperature for 10 minutes, instead of homogenization.

Microfiltrations according to the earlier process were all performed with Millipore Pellicon 2 systems. For better practicability and to avoid time-consuming cleaning procedures, the microfiltrations in this Example were performed with the following equipment:

| Filtration | | Filter System | Cartouche |
|---|---|---|---|
| 0.2 | μm filtration | Millipore Pellicon | Durapore 0.2μ, C-screen |
| | | 2 | |
| 0.1 | μm filtration | One way filter | Millipack 200, 0.1 μm |
| 1000 | kDa filtration | Millipore Pellicon | Biomax 1000 kDa, V-Screen |
| | | 2 | |
| 0.2 | μm filtration | One way filter | Millipack 200, 0.2 μm |

In addition, phenol was added, for stabilization of the extract. The amount of added phenol was 6.0-8.0 mg/ml. It was added after the 1000 kDa filtration. After the addition, the suspension was stirred for approximately 10 minutes, until all phenol was dissolved.

The endotoxin level was low in each case.

### Example 7

### Comparative Example

Example 1 was repeated, except that the last two filtration steps were omitted.

Residue of bacterial endotoxins in the final filtrate: 1917 EU/ml.

### Example 8

### Comparative Example

Example 1 was repeated, except that the last filtration step was omitted.

Residue of bacterial endotoxins in the final filtrate: 1556 EU/ml

### Example 9

### Comparative Example

Example 1 was repeated, except that no ricinus oil was added, and the last two filtration steps were omitted.

Residue of bacterial endotoxins in the final filtrate: 3095 EU/ml.

### Example 10

### Comparative Example

Example 1 was repeated, except that no ricinus oil was added, and the last filtration step was omitted.

Residue of bacterial endotoxins in the final filtrate: 4839 EU/ml.

### Example 11

### Comparative Example

Example 1 was repeated, except that no ricinus oil was added.

Residue of bacterial endotoxins in the final filtrate: 2068 EU/ml.

1. A process for purifying as aqueous composition including a water-soluble contaminant having lipid groups, which comprises contacting the composition with a lipophilic component that forms a complex with the contaminant; a first removal step, of material having a size larger than the complex; and a second removal step, of the complex.

2. A process according to claim 1, wherein the composition is a plant extract.

3. A process according to claim 2, wherein the plant extract is an aqueous extract.

4. A process according to claim 2, wherein the plant is chamomile.

5. A process according to claim 4, wherein the extract is of the flores tubiformis.

6. A process according to claim 1, wherein the water-soluble contaminant is a carbohydrate having also protein groups.

7. A process according to claim 6, wherein the contaminant is an endotoxin.

8. A process according to claim 1, wherein the lipophilic component is an oil.

9. A process according to claim 1, wherein the first removal step comprises microfiltration.

10. A process according to claim 9, wherein the microfiltration uses a filter having a pore size of at least 0.1 μm.

11. A process according to claim 1, wherein the second removal step comprises ultrafiltration.

12. A process according to claim 1, wherein
the composition is a plant extract,
the plant extract is an aqueous extract,
the water-soluble contaminant is a carbohydrate having also protein groups,
the lipophilic component is an oil, and
the first removal step comprises microfiltration.

13. A process according to claim 12, wherein the contaminant is an endotoxin.

14. A process according to claim 13, wherein the plant extract is chamomile extract.

15. A process according to claim 12, wherein the second removal step comprises ultrafiltration.

16. A process according to claim 12, wherein the microfiltration uses a filter having a pore size of at least 0.1 μm.

17. A process according to claim 12, wherein the plant extract is chamomile extract and the microfiltration uses a filter having a pore size of at least 0.1 μm.

18. A process according to claim 17, wherein the contaminant is an endotoxin.

19. A process according to claim 17, wherein the extract is of the flores tubiformis.
